(19) United States
(12) Patent Application Publication
(10) Pub. No.: US 2025/0161571 A1
(43) Pub. Date: May 22, 2025

(54) Syringe With Minimal Internal Dead Volume

(71) Applicant: Becton, Dickinson and Company, Franklin Lakes, NJ (US)

(72) Inventors: Paul Paia Marici, Piscataway, NJ (US); Suzanne Ferreri, Glen Rock, NJ (US)

(21) Appl. No.: 18/832,532

(22) PCT Filed: Feb. 8, 2023
(86) PCT No.: PCT/US23/62165

§ 371 Date: Jul. 24, 2024

Related U.S. Application Data
(60) Provisional application No. 63/308,131, filed on Feb. 9, 2022.

(51) Int. Cl. A61M 5/31; A61M 5/32

(52) U.S. Cl. CPC A61M 5/3134; A61M 5/3293

(57) ABSTRACT

A syringe body having a housing and a cannula assembly. The housing includes a proximal end, a distal end, and a sidewall extending between the proximal and distal end, the sidewall defining a chamber adapted to receive a medicament, and a fitting extending from the distal end of the housing and comprising an open proximal end, an open distal end, and a sidewall extending therebetween, an interior surface of the sidewall defining a passageway. The cannula assembly includes a hub and a cannula inserted in the passageway, and the hub is sized and shaped to substantially fill the passageway of the fitting. Also, a syringe including the syringe body, a plunger comprising a plunger rod, and a stopper attached to a distal end of the plunger rod and moveably received in the chamber of the housing of the syringe body.

### CROSS-REFERENCE TO RELATED APPLICATION

The present application claims priority to U.S. Provisional Application Ser. No. 63/308,131, entitled “Syringe with Minimal Internal Dead Volume”, filed Feb. 9, 2022, the entire disclosure of which is hereby incorporated by reference in its' entirety.

### BACKGROUND OF THE INVENTION

### Field of the Invention

The present invention is directed to a syringe body having a housing having a cannula fitting and a cannula assembly with minimal area provided in the cannula fitting for the retention of medicament after an injection, and a syringe including the inventive syringe body and a plunger.

### Description of Related Art

Fittings 1 and hubs 3 for attaching cannulas 5 to syringe bodies 7 have been standardized under ISO-80369-7:2016, revised in 2021 entitled “Small-bore connectors for liquids and gases in healthcare applications-Part 7: Connectors for intravascular or hypodermic applications” and include luer slip fittings 1a and luer lock fittings 1b (FIGS. 1 and 2). As can be seen in FIGS. 1 and 2, in prior art syringe bodies 7a, 7b having luer slip fittings 1a or luer lock fittings 1b that utilize cannula assemblies having hubs 3 that fit over the male luer taper, an area in which a significant amount of medicament is retained after an injection using the syringe is present between the stopper 9 and the proximal end of the cannula 5. This volume of medicament as well as the medicament that is retained in the bore of the cannula 5 is referred to as the dead volume. Thus, the prior art cannulas have a significant dead volume which results in a significant amount of medicament being wasted with each injection and a reduced number of doses being able to be obtained from a medicament vial having a specific volume of medicament, thereby increasing packaging and storage costs. Therefore, it would be advantageous to have a syringe with less dead volume in order to reduce the amount of medicament that is wasted and to reduce packaging and storage costs, especially when the medicament must be refrigerated.

### SUMMARY OF THE INVENTION

The present invention is directed to a syringe body comprising a housing comprising a proximal end, a distal end, and a sidewall extending between the proximal and distal end, the sidewall defining a chamber adapted to receive a medicament, and a fitting extending from the distal end of the housing and comprising an open proximal end, an open distal end, and a sidewall extending therebetween, an interior surface of the sidewall defining a passageway; and a cannula assembly comprising a hub and a cannula inserted in the passageway, wherein the hub is sized and shaped to substantially fill the passageway of the fitting.

The interior surface of the sidewall of the fitting may taper in a distal direction, radially inward toward a longitudinal axis of the syringe body, such that a diameter of the passageway at the open proximal end of the fitting is greater than a diameter of the passageway at the open distal end of the fitting, and the hub may have an exterior surface that is tapered to correspond to the taper of the interior surface of the sidewall of the fitting, such that a diameter of the hub at a proximal end of the hub is greater than a diameter of the hub at a distal end of the hub. An exterior surface of the hub may form an interference fit with the interior surface of the sidewall of the fitting to create a fluid tight seal between the hub and the fitting, wherein the fluid tight seal prevents any medicament provided in the chamber from entering into the interface between the exterior surface of the hub and the interior surface of the sidewall of the fitting. The hub may be press fit into the passageway of the fitting, and/or the hub may be held in the passageway by outward radial forces exerted by the hub on the sidewall of the fitting and/or frictional engagement between an exterior surface of the hub and the interior surface of the sidewall of the fitting. A proximal end of the hub may be flush with the proximal end of the fitting.

The hub may further comprises one or more protrusions extending from an exterior surface of the hub. The one or more protrusions may extend around a circumference of the exterior surface of the hub. A protrusion may be positioned a distance from the proximal end of the hub, wherein a portion of the hub between the proximal end of the hub and the protrusion creates an interference fit between an exterior surface of the hub and the interior surface of the sidewall of the fitting that provides a fluid tight seal between the hub and the fitting. The one or more protrusions may have a cross-section that is substantially triangular. A first surface of the protrusion may extend radially outward from the hub in a substantially horizontal direction perpendicular to a longitudinal axis of the hub, and a second surface may extend at an angle from an outer edge of the first surface to the exterior surface of the hub in a radially inward direction toward the longitudinal axis of the hub.

The hub may further comprise one or more grooves extending into an exterior surface of the hub. The one or more grooves may extend around a circumference of the exterior surface of the hub. The one or more grooves may be rounded having a curved inner edge, and may have a semicircular cross section. Adhesive may be provided in the one or more grooves and at least partially between the hub and the interior surface of the sidewall of the fitting.

The hub may further comprise a substantially cylindrical central bore adapted to receive the cannula, wherein the bore extends through the hub from the open proximal end of the hub to the open distal end of the hub and the cannula is received in the central bore such that a portion of the cannula extends from the distal end of the hub. The central bore may include a stop for limiting a distance that the cannula is inserted into the central bore. The stop may be an annular ledge extending around the circumference of and radially inward from an interior surface of the central bore reducing the diameter of the central bore in the area of the ledge. The central bore may further comprise an enlarged portion having a proximal end coinciding with the proximal end of the hub and a distal end. An interior surface of the enlarged portion may taper in a distal direction, radially inward toward a longitudinal axis of the hub, such that a diameter of the enlarged portion at the proximal end of the enlarged portion is greater than a diameter of the enlarged portion at the distal end of the enlarged portion.

The distal end of the fitting may include a stop for keeping a distal end of the hub from extending out of the distal end of the fitting. The stop may be an annular ledge extending around a circumference of and radially inward from the interior surface of the sidewall of the fitting and reducing the diameter of the passageway in the area of the ledge.

The fitting may be a male luer taper. The housing may further comprises a collar having a proximal end, a distal end, and a sidewall extending therebetween, the collar extending longitudinally from the distal end of the housing and the sidewall surrounding the fitting. Threads may be provided on an interior surface of the sidewall of the collar.

The housing may further comprise a flange extending radially outward from the proximal end of the housing in a direction perpendicular to a longitudinal axis of the syringe body.

The present invention is also directed to a syringe comprising a syringe body having any or all of the features discussed above, a plunger comprising a plunger rod, and a stopper attached to a distal end of the plunger rod and moveably received in the chamber of the housing of the syringe body.

### BRIEF DESCRIPTION OF THE DRAWINGS

FIG. 1 is a cross-sectional view of a prior art syringe having a luer slip connection;

FIG. 2 is a cross-sectional view of a prior art syringe having a luer lock connection;

FIG. 3 is a cross-sectional view of a first embodiment shown with Luer Slip syringe according to the invention;

FIG. 3A is an enlarged cross-sectional view of area 3A of FIG. 3 including a cannula shield.

FIG. 4 is a cross-sectional view of a second embodiment shown with Luer Lock syringe according to the invention;

FIG. 5 is a perspective view of a first embodiment of a hub according to the invention;

FIG. 6 is a cross-sectional view along line 6-6 of the hub of FIG. 5;

FIG. 7 is a cross-sectional view of the hub of FIG. 5 inserted in the fitting of the inventive syringe body;

FIG. 8 is a perspective view of a second embodiment of a hub according to the invention;

FIG. 9 is a cross-sectional view along line 9-9 of the hub of FIG. 8;

FIG. 9A is an enlarged cross-sectional view of area 9A of FIG. 9;

FIG. 10 is a cross-sectional view of the hub of FIG. 8 inserted in the fitting of the inventive syringe body;

FIG. 11 is a perspective view of a an embodiment of a hub according to the invention;

FIG. 12 is a cross-sectional view along line 12-12 of the hub of FIG. 11;

FIG. 12A is an enlarged cross-sectional view of area 12A of FIG. 12; and

FIG. 13 is a cross-sectional view of the hub of FIG. 11 inserted in the fitting of the inventive syringe body.

### DESCRIPTION OF THE INVENTION

As used herein, unless otherwise expressly specified, all numbers such as those expressing values, ranges, amounts or percentages may be read as if prefaced by the word “about”, even if the term does not expressly appear. Any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1. Plural encompasses singular and vice versa. When ranges are given, any endpoints of those ranges and/or numbers within those ranges can be combined with the scope of the present invention. “Including”, “such as”, “for example” and like terms means “including/such as/for example but not limited to”.

For purposes of the description hereinafter, spatial orientation terms, as used, shall relate to the referenced embodiment as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

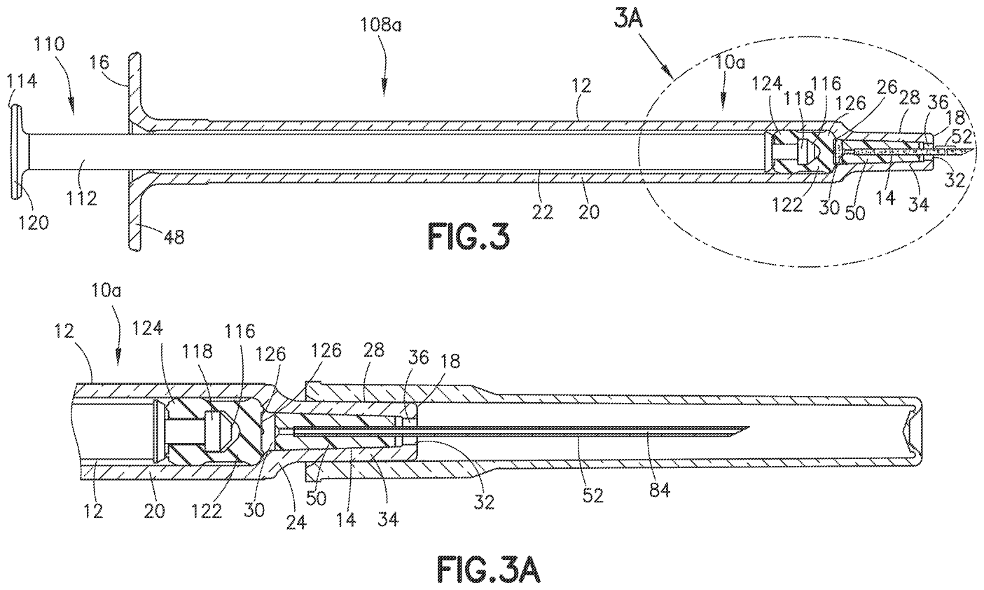

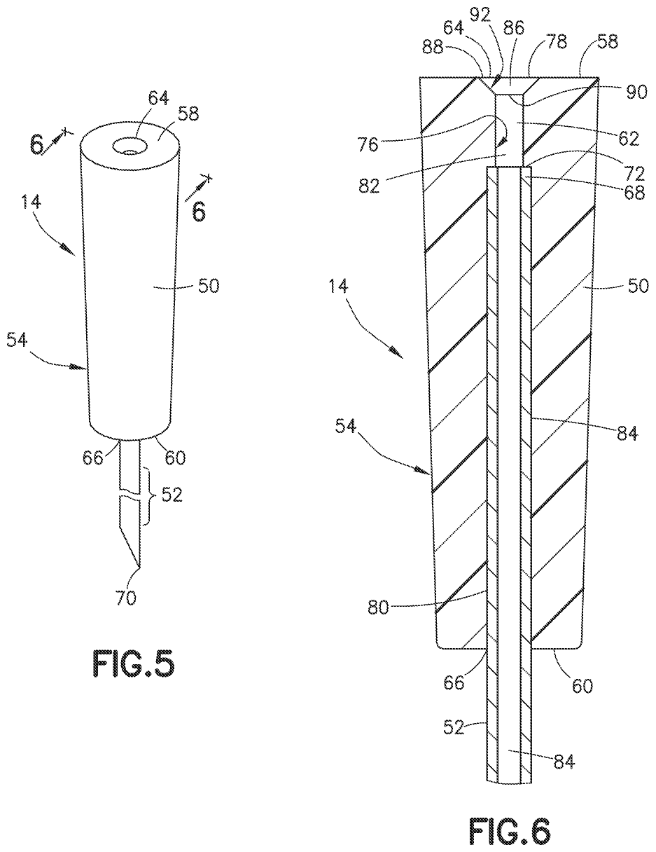

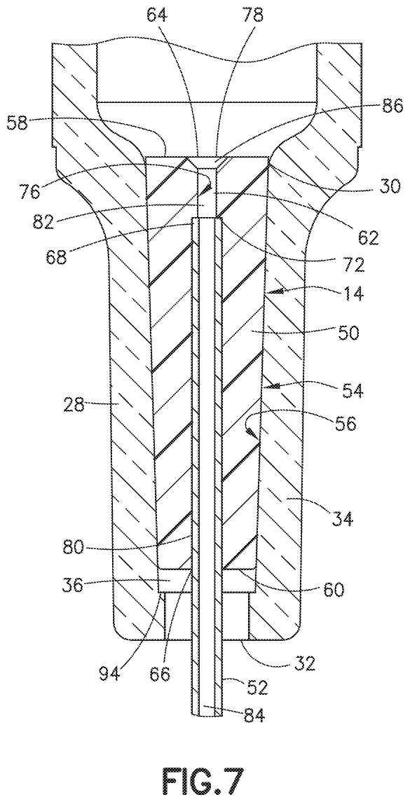

As shown in FIGS. 3, 3A, and 4-7, the present invention is directed to a syringe body 10 comprising a housing 12 and a cannula insert 14.

The housing 12 comprises a proximal end 16, a distal end 18, and a sidewall 20 extending between the proximal end 16 and distal end 18. The sidewall 20 defines a chamber 22 adapted to receive a medicament. The chamber 22 may be substantially cylindrical. A bottom surface 24 extends inwardly in a radial direction from the sidewall 20 of the housing 12 at the proximal end 16 of the housing 12. An opening 26 is provided in the bottom surface 24 and is in fluid communication with a fitting 28 that extends in the longitudinal direction from the distal end 18 of the housing 12.

The fitting 28 comprises an open proximal end 30, an open distal end 32, and a sidewall 34 extending therebetween. The sidewall 34 defines a passageway 36, and the open proximal end 30 of the fitting 28 is in fluid communication with the opening 26 in the bottom surface 24 of the housing 12 and the chamber 22 of the housing 12 and in fluid communication with the open distal end 32 via the passageway 36. The passageway 36 tapers radially inward toward the longitudinal axis L of the syringe body 10 in a distal direction, such that the diameter of the passageway 36 at the open proximal end 30 of the fitting 28 is greater than the diameter of the passageway 36 at the open distal end 32 of the fitting 28. The passageway 36 may have a truncated conical shape. The fitting 28 may be a male luer taper, which is often referred to as a luer-slip fitting and may comply with ISO-80369-7:2016, revised in 2021 entitled “Small-bore connectors for liquids and gases in healthcare applications—Part 7: Connectors for intravascular or hypodermic applications”, incorporated herein by reference.

The housing 12 may further comprise a collar 38 having a proximal end 40, a distal end 42, and a sidewall 44 extending therebetween. The collar 38 extends longitudinally from the distal end 18 of the housing 12 and surrounds the fitting 28. Threads 46 may be included on the interior surface of the sidewall 44 of the collar 38. The collar 38 in combination with the fitting 28 may be a luer lock.

A flange 48 may extend radially outward from the proximal end 16 of the housing 12 in a direction perpendicular to the longitudinal axis L of the syringe body 10.

The cannula insert 14 comprises a hub 50 and a cannula 52.

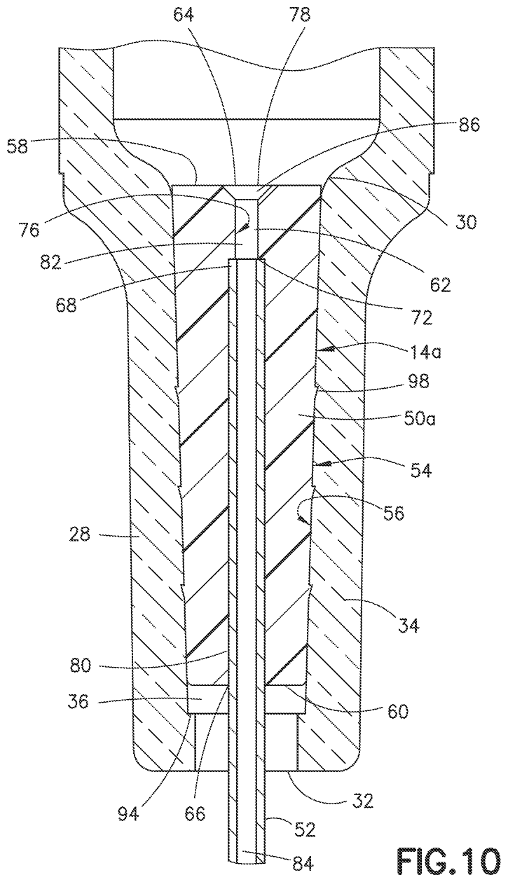

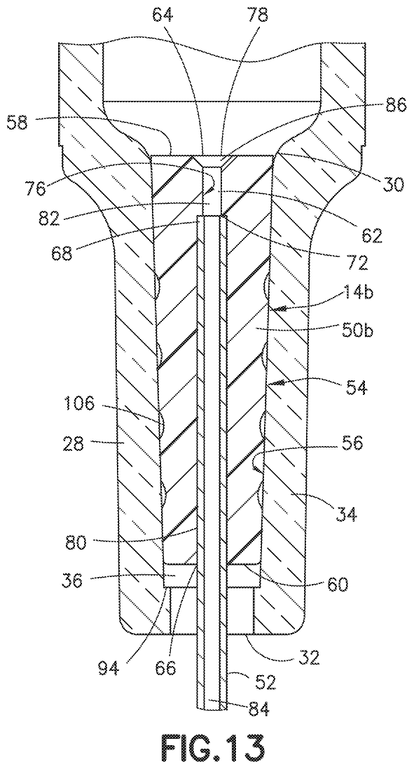

The hub 50 and its interaction with the fitting 28 are shown in detail in FIGS. 5-13. The hub 50 has an exterior surface 54 that is tapered to correspond to the taper of the interior surface 56 of the sidewall 34 of the fitting 28. The exterior surface 54 of the hub 50 tapers in a distal direction, radially inward toward the longitudinal axis L1 of the hub 50, such that the diameter of the hub 50 at the proximal end 58 of the hub 50 is greater than the diameter of the hub 50 at the distal end 60 of the hub 50.

A substantially cylindrical bore 62 adapted to receive the cannula 52 extends through the hub 50 from the proximal end 58 of the hub 50 to the distal end 60 of the hub 50. The bore 62 creates a proximal opening 64 in the proximal end 58 of the hub 50 and a distal opening 66 in the distal end 60 of the hub 50. The bore 62 may extend along the longitudinal axis L1 of the hub 50.

The cannula 52 has a proximal end 68 and a distal end 70 with a beveled point for piercing the tissue of a patient. The cannula 52 is received in the bore 62 such that a portion of the distal end 70 of the cannula 52 extends from the distal end 60 of the hub 50.

The bore 62 may include a stop 72 for limiting the distance that the cannula 52 may be inserted into the bore 62. The stop 72 may be an annular ledge extending around the circumference of and radially inward from the interior surface 76 of the bore 62 reducing the diameter of the bore 62 in the area of the stop 72. The reduced diameter of the bore 62 of the hub 50 may extend from the stop 72 to the proximal end 78 of the bore 62 such that the bore 62 has a distal portion 80 having a first diameter and a proximal portion 82 having a second diameter where the second diameter is smaller than the first diameter. The radial distance which the stop 72 extends into the bore 62 is a distance that allows the stop 72 to engage the proximal end 68 of the cannula 52 to limit the distance that the cannula 52 can be received in the bore 62 of the hub 50 without blocking the bore 84 of the cannula 52. In certain embodiments, the cannula 52 may extend the entire length to proximal end 58 of the hub 50, in which the limitation of the distance of insertion into bore 62 is eliminated.

The bore 62 may further comprise an enlarged portion 86 having a proximal end 88 coinciding with the proximal end 58 of the hub 50 and a distal end 90. The enlarged portion 86 may have a tapered truncated conical shape. The interior surface 92 of the enlarged portion 92 may taper in a distal direction, radially inward toward the longitudinal axis L1 of the hub 50, such that the diameter of the enlarged portion 86 at the proximal end 88 of the enlarged portion 86 is greater than the diameter of the interior surface 92 of the enlarged portion 86 at the distal end 90 of the enlarged portion 86. When an enlarged portion 86 is provided for the bore 62, the diameter of the proximal opening 64 of the bore 62 is larger than the distal opening 66 of the bore 62.

The hub 50 may be made of any suitable material including polypropylene, polyethylene or other medical grade resins approved to be in contact with medication and may be made using an injection molding process.

The cannula 52 may be fixed within the hub 50 by insert or over molding during the molding process used to produce the hub 50 or may be inserted into the bore 62 of the hub 50 after the production of the hub 50. Glue or adhesive may be provided to hold the cannula 52 within the bore 62 of the hub 50. One or more grooves (not shown) in the interior surface 76 of the bore 62 of the hub 50 extending around the circumference or in the longitudinal direction of the hub 50 may be provided to allow for glue or adhesive to be used to secure the cannula 52 within the hub 50.

The hub 50 of the cannula insert 14 is received in the passageway 36 of the fitting 28 of the housing 12 such that at least a portion of the cannula 52 extending from the distal end 60 of the hub 50 extends from the distal end 18 of the housing 12. The hub 50 is sized and shaped to substantially fill the passageway 36 of the fitting 28. Correspondence between the shape of the exterior surface 54 of the hub 50 and the interior surface 56 of the sidewall 34 of the fitting 28 provides an interference fit between the hub 50 and the fitting 28 to create a fluid tight seal between the hub 50 and the fitting 28. The fluid tight seal prevents any medicament provided in the chamber 22 from entering into the interface between the exterior surface 54 of the hub 50 and the interior surface 56 of the sidewall 34 of the fitting 28. The hub 50 may be press fit into the passageway 36 of the fitting 28 and may be held in the passageway 36 by outward radial forces exerted by the hub 50 on the sidewall 34 of the fitting 28 created by the press fit and/or frictional engagement between the exterior surface 54 of the hub 50 and the interior surface 56 of the sidewall 34 of the fitting 28. The proximal end 58 of the hub 50 may be flush with the open proximal end 30 of the fitting 28. The distal end 60 of the hub 50 may be flush with the open distal end 32 of the fitting 28 or a gap may be provided between the distal end 60 of the hub 50 and the open distal end 32 of the fitting 28.

The open distal end 32 of the fitting 28 may optionally include a stop 94 for keeping the distal end 60 of the hub 50 from extending out of the open distal end 32 of the fitting 28. The stop 94 may be an annular ledge extending around the circumference of and radially inward from the interior surface 56 of the sidewall 34 of the fitting 28 and reducing the diameter of the passageway 36 in the area of the stop 94. The reduced diameter of the passageway 36 of the fitting 28 may extend from the stop 94 to the distal end 32 of the fitting 28. The radial distance which the stop 94 extends into the bore 62 is a distance that allows the stop 94 to engage the distal end 60 of the hub 50 to limit the distance that the hub 50 can be received in the passageway 36 of the fitting 28.

With the hub 50 received and secured in the passageway 36 of the fitting 28, a fluid passageway is provided from the chamber 22 of the housing 12 through the proximal portion 82 of the bore 62 of the hub 50, and through the bore 84 of the cannula 52. If present, the enlarged portion 86 of the bore 62 of the hub 50 directs the fluid into the narrowed proximal portion 82 of the bore 62 and/or into the bore 84 of the cannula 52 and reduces the possibility of bubbles entering the bore 62 of the hub 50 and/or the bore 84 of the cannula 52.

The hub 50 may be provided with additional features to allow the hub 50 to be securely held in the passageway 36 of the fitting 28 while providing a fluid tight seal between the hub 50 and the fitting 28.

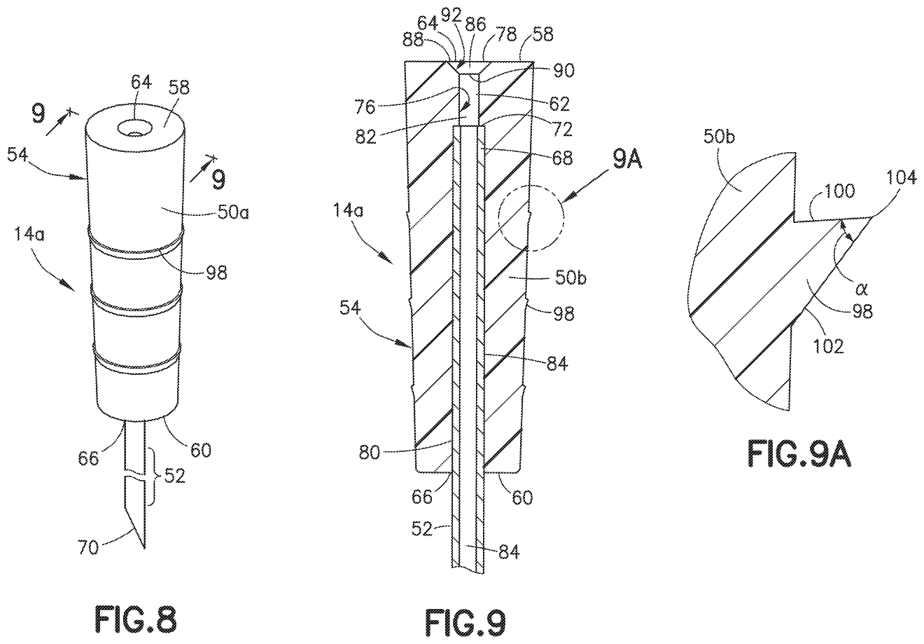

In one embodiment (FIGS. 8, 9, 9A, and 10), the hub 50a of the cannula insert 14a may have one or more protrusions 98 extending from the exterior surface 54 of the hub 50a. The protrusion 98 may extend around the circumference of the exterior surface 54 of the hub 50a. The protrusion 98 is positioned a distance from the proximal end 58 of the hub 50a. The portion of the hub 50a between the proximal end 58 of the hub 50a and the protrusion 98 creates an interference fit between the exterior surface 54 of the hub 50a and the interior surface 56 of the sidewall 34 of the fitting 28 that provides a fluid tight seal between the hub 50a and the fitting 28.

The protrusion 98 may have a cross-section that is substantially triangular. A first surface 100 of the protrusion 98 may extend radially outward from the hub 50a in a substantially horizontal direction perpendicular to the longitudinal axis L1 of the hub 50a. A second surface 102 extends at an angle from the outer edge 104 of the first surface 100 to the exterior surface 54 of the hub 50a in a radially inward direction toward the longitudinal axis L1 of the hub 50a. An angle α between the first surface 100 and the second surface 102 of the protrusion 98 is less than 90°.

The second surface 102 allows the hub 50a to be easily inserted into the passageway 36 of the fitting 28 by providing an angled surface that interacts with the interior surface 56 of the sidewall 34 of the fitting 28. The hub 50a is then locked within the passageway 36 of the fitting 28 by the interference fit between the exterior surface 54 of the proximal end 58 of the hub 50a and the interior surface 56 of the sidewall 34 of the fitting 28 and by the first surface 100 of the protrusion 98 which interacts with the interior surface 56 of the sidewall 34 of the fitting 28 to resist any proximally directed force placed on the hub 50a or the cannula 52 during use.

Alternatively, the protrusion may be rounded, having a curved outer edge and may have a semicircular cross section, may have a square or rectangular cross-section having opposing surfaces extending radially outward from the hub in a substantially horizontal direction perpendicular to the longitudinal axis of the hub and connected by a surface extending between the outer ends of the surfaces in a substantially vertical direction parallel to the longitudinal axis of the hub, may have a polygonal cross-section having opposing surfaces extending radially outward from the hub in a substantially horizontal direction perpendicular to the longitudinal axis of the hub, where the proximal substantially horizontal surface has a larger radial width than the radial width of the distal substantially horizontal surface and the opposing substantially horizontal surfaces are connected by a surface extending at an angle from the outer edge of the proximal substantially horizontal surface to the outer edge of the distal substantially horizontal surface in a radially inward direction toward the longitudinal axis of the hub.

If more than one protrusion 98 is provided, the protrusions 98 may have the same cross-sectional size and shape or individual protrusions may have different cross-sectional sizes and/or shapes. The protrusions 98 may be spaced equally along the exterior surface 54 of the hub 50a or the distance along the exterior surface 54 of the hub 50a between adjacent protrusions 98 may vary.

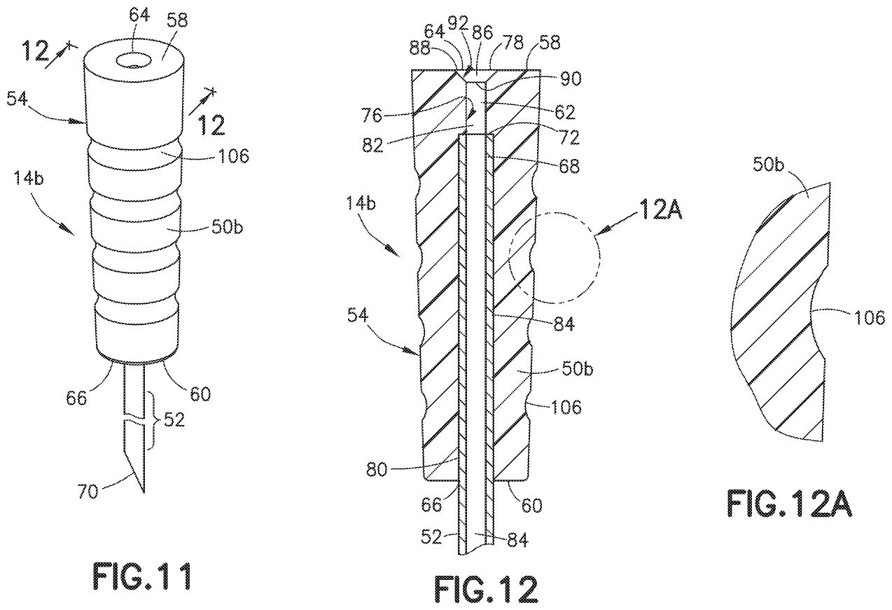

In another embodiment (FIGS. 11, 12, 12A, and 13), the hub 50b of the cannula insert 14b may have one or more grooves 106 extending into the exterior surface 54 of the hub 50b. The groove 106 may extend around the circumference of the exterior surface 54 of the hub 50b. The groove 106 may be positioned a distance from the proximal end 58 of the hub 50b. The portion of the hub 50b between the proximal end 58 of the hub 50b and the groove 106 creates an interference fit between the exterior surface 54 of the hub 50b and the interior surface 56 of the sidewall 34 of the fitting 28 that provides a fluid tight seal between the hub 50b and the fitting 28.

The groove 106 may be rounded having a curved inner edge and may have a semicircular cross section.

During assembly, glue, epoxy, or another suitable adhesive may be provided in the groove 106 where it can squeeze out of the groove 106 when the hub 50b is press fit into the passageway 36 of the fitting 28, creating an adhesive bond between the hub 50b and the interior surface 56 of the sidewall 34 of the fitting 28.

The groove 106 can be provided in combination with the above described protrusion 98.

Alternatively, the groove may have a square or rectangular cross-section having opposing surfaces extending radially inward into the hub in a substantially horizontal direction perpendicular to the longitudinal axis of the hub and connected by a surface extending between the inner ends of the surfaces in a substantially vertical direction parallel to the longitudinal axis of the hub, may have a polygonal cross-section having opposing surfaces extending radially inward into the hub in a substantially horizontal direction perpendicular to the longitudinal axis of the hub, where one of the proximal substantially horizontal surface and the distal substantially horizontal surface has a larger radial width than the other and the opposing substantially horizontal surfaces are connected by a surface extending at an angle from the inner edge of one of the substantially horizontal surfaces to the inner edge of the other substantially horizontal surface.

If more than one groove 106 is provided, the grooves 106 may have the same cross-sectional size and shape or individual grooves 106 may have different cross-sectional sizes and/or shapes. The grooves 106 may be spaced equally along the exterior surface 54 of the hub 50b or the distance along the exterior surface 54 of the hub 50b between adjacent grooves 106 may vary.

In another embodiment, the exterior surface of the hub and/or the interior surface of the sidewall of the fitting may be textured in order to increase the friction between the surface of the exterior surface of the hub and the interior surface of the sidewall of the fitting. For example, the exterior surface of the hub and/or the interior surface of the sidewall of the fitting could have a crepe finish, a cross-hatched pattern, or a rough surface finish provided through injection molding. The texture on the exterior surface of the hub and/or the interior surface of the sidewall of the fitting can be provided in combination with the above-described protrusion and/or groove.

The present invention is also directed to a syringe 108 comprising the above-described syringe body 10 and a plunger 110.

The plunger 110 comprises a plunger rod 112 having a proximal end 114 and a distal end 116, an engagement portion 118 extending from the distal end 116 of the plunger rod 112, and a thumb pad 120 extending radially outward from the proximal end 114 of the plunger rod 112 in a direction substantially perpendicular to the longitudinal axis of the plunger rod 112. At least a portion of the plunger rod 112 and the engagement portion 118 are contained within the chamber 22 of the housing 12. The engagement portion 118 is adapted for attachment to a stopper 122. The plunger 110 is movable with respect to the syringe body 10 such that a distal force applied to the plunger 110 while holding the syringe body 10 stationary causes the engagement portion 118 of the plunger 110 and the stopper 122 attached thereto to be displaced in a distal direction within the chamber 22 of the housing 12, and a proximal force applied to the plunger 110 while holding the syringe body 10 stationary causes the engagement portion 118 of the plunger 110 and the stopper 122 attached thereto to be displaced in a proximal direction within the chamber 22 of the housing 12.

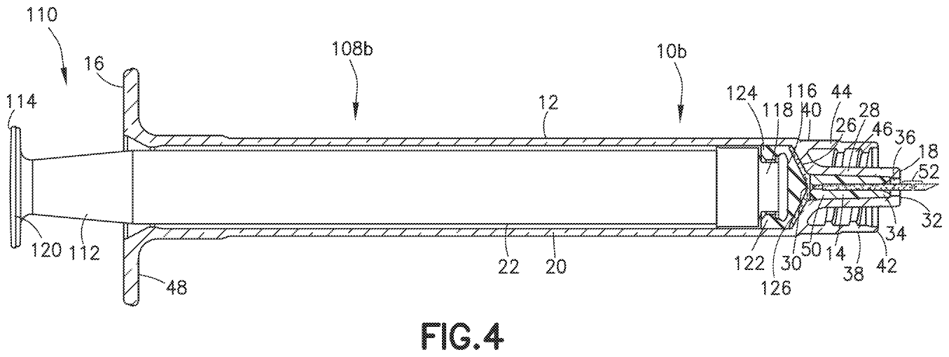

The stopper 122 has a proximal end 124 attached to the engagement portion 118 of the plunger 110 and a distal end 126. The distal end 126 of the stopper 122 may have a flat surface as shown in FIGS. 3 and 3A or may have a conical surface as shown in FIG. 4.

The syringe body 10 and the syringe 108 may further comprise a cannula shield 128 covering the portion of the cannula 52 extending from the fitting 28 (FIG. 3A). The cannula shield 128 may comprise an open proximal end 130, a closed distal end 132, and a sidewall 134 extending therebetween. The sidewall 134 defines a cavity 136 adapted to receive at least a portion of the fitting 28 and the portion of the cannula 52 extending from the open distal end 32 of the fitting 28. The interior surface 138 of the sidewall 134 of the proximal portion of the cannula shield 128 may have a size and shape corresponding to the exterior surface 140 of the fitting 28. An interference fit may be provided between the interior surface 138 of the sidewall 134 of the proximal portion of the cannula shield 128 and the exterior surface 140 of the fitting 28, thereby securing the cannula shield 128 to the fitting 28.

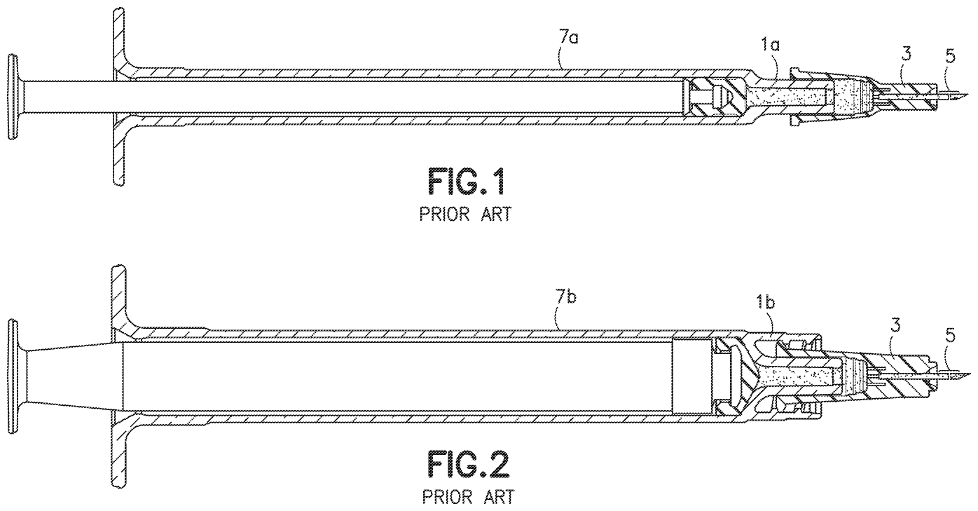

As can be seen in FIGS. 1 and 2, in prior art syringe bodies having luer slip or luer lock connections that utilize cannula assemblies having hubs that fit over the male luer taper, an area in which a significant amount of medicament is retained after an injection using the syringe is present between the stopper and the proximal end of the cannula. This volume of medicament, as well as the medicament that is retained in the bore of the cannula, is referred to as the dead volume. Thus, the prior art cannulas have a significant dead volume which results in a significant amount of medicament being wasted with each injection.

As can be seen in FIGS. 3 and 4 in the inventive syringe body, there is very little space between the stopper and the proximal end of the cannula resulting in significantly less dead volume and up to a 20-40% decrease in wasted medicament. By reducing the dead volume and thereby reducing the wasted medicament, several advantages can be realized. First, the cost of each injection is reduced because less medicament is wasted. Second, for a vial holding a defined amount of medicament, packaging and storage costs can be reduced because more doses can be obtained from the defined amount of medicament. Third, the syringe body of the inventive syringe can advantageously be produced using available molds made for syringes having luer slip or luer lock connections.

Whereas particular aspects of this invention have been described above for purposes of illustration, it will be evident to those skilled in the art that numerous variations of the details of the present invention may be made without departing from the invention.

1. A syringe body comprising:
a housing comprising:
a proximal end, a distal end, and a sidewall extending between the proximal and distal end, the sidewall defining a chamber adapted to receive a medicament, and
a fitting extending from the distal end of the housing and comprising an open proximal end, an open distal end, and a sidewall extending therebetween, an interior surface of the sidewall defining a passageway; and
a cannula assembly comprising a hub and a cannula inserted in the passageway, wherein the hub is sized and shaped to substantially fill the passageway of the fitting.

2. A syringe body according to claim 1, wherein the interior surface of the sidewall of the fitting tapers in a distal direction, radially inward toward a longitudinal axis of the syringe body, such that a diameter of the passageway at the open proximal end of the fitting is greater than a diameter of the passageway at the open distal end of the fitting, and the hub has an exterior surface that is tapered to correspond to the taper of the interior surface of the sidewall of the fitting, such that a diameter of the hub at a proximal end of the hub is greater than a diameter of the hub at a distal end of the hub.

3. A syringe body according to claim 1, wherein an exterior surface of the hub forms an interference fit with the interior surface of the sidewall of the fitting to create a fluid tight seal between the hub and the fitting, wherein the fluid tight seal prevents any medicament provided in the chamber from entering into the interface between the exterior surface of the hub and the interior surface of the sidewall of the fitting.

4. A syringe body according to claim 1, wherein the hub is press fit into the passageway of the fitting.

5. A syringe body according to claim 1, wherein the hub is held in the passageway by outward radial forces exerted by the hub on the sidewall of the fitting and/or frictional engagement between an exterior surface of the hub and the interior surface of the sidewall of the fitting.

6. A syringe body according to claim 1, wherein a proximal end of the hub is flush with the proximal end of the fitting.

7. A syringe body according to claim 1, wherein the hub further comprises one or more protrusions extending from an exterior surface of the hub.

8. A syringe body according to claim 7, wherein one or more protrusions extend around a circumference of the exterior surface of the hub.

9. A syringe body according to claim 7, wherein a protrusion is positioned a distance from the proximal end of the hub, wherein a portion of the hub between the proximal end of the hub and the protrusion creates an interference fit between an exterior surface of the hub and the interior surface of the sidewall of the fitting that provides a fluid tight seal between the hub and the fitting.

10. A syringe body according to claim 7, wherein the one or more protrusions have a cross-section that is substantially triangular.

11. A syringe body according to claim 10, wherein a first surface of the protrusion extends radially outward from the hub in a substantially horizontal direction perpendicular to a longitudinal axis of the hub, and a second surface extends at an angle from an outer edge of the first surface to the exterior surface of the hub in a radially inward direction toward the longitudinal axis of the hub.

12. A syringe body according to claim 1, wherein the hub further comprises one or more grooves extending into an exterior surface of the hub.

13. A syringe body according to claim 12, wherein one or more grooves extend around a circumference of the exterior surface of the hub.

14. A syringe body according to claim 12, wherein the one or more grooves are rounded having a curved inner edge.

15. A syringe body according to claim 14, wherein the one or more grooves have a semicircular cross section.

16. A syringe body according to claim 12, wherein adhesive is provided in the one or more grooves and at least partially between the hub and the interior surface of the sidewall of the fitting.

17. A syringe body according to claim 1, wherein the hub further comprises a substantially cylindrical central bore adapted to receive the cannula, wherein the bore extends through the hub from the open proximal end of the hub to the open distal end of the hub, and the cannula is received in the central bore such that a portion of the cannula extends from the distal end of the hub.

18. A syringe body according to claim 17, wherein the central bore includes a stop for limiting a distance that the cannula is inserted into the central bore.

19. A syringe body according to claim 18, wherein the stop is an annular ledge extending around the circumference of and radially inward from an interior surface of the central bore reducing the diameter of the central bore in the area of the ledge.

20. A syringe body according to claim 17, wherein the central bore further comprises an enlarged portion having a proximal end coinciding with the proximal end of the hub and a distal end.

21. A syringe body according to claim 20, wherein an interior surface of the enlarged portion tapers in a distal direction, radially inward toward a longitudinal axis of the hub, such that a diameter of the enlarged portion at the proximal end of the enlarged portion is greater than a diameter of the enlarged portion at the distal end of the enlarged portion.

22. A syringe body according to claim 1, wherein the distal end of the fitting includes a stop for keeping a distal end of the hub from extending out of the distal end of the fitting.

23. A syringe body according to claim 22, wherein the stop is an annular ledge extending around a circumference of and radially inward from the interior surface of the sidewall of the fitting and reducing the diameter of the passageway in the area of the ledge.

24. A syringe body according to claim 1, wherein the fitting is a male luer taper.

25. A syringe body according to claim 1, wherein the housing further comprises a collar having a proximal end, a distal end, and a sidewall extending therebetween, the collar extending longitudinally from the distal end of the housing and the sidewall surrounding the fitting.

26. A syringe body according to claim 25, wherein threads are provided on an interior surface of the sidewall of the collar.

27. A syringe body according to claim 1, wherein the housing further comprises a flange extending radially outward from the proximal end of the housing in a direction perpendicular to a longitudinal axis of the syringe body.

28. A syringe comprising:
a syringe body according to claim 1;
a plunger comprising a plunger rod; and
a stopper attached to a distal end of the plunger rod and moveably received in the chamber of the housing of the syringe body.
